(19) United States
(12) Patent Application Publication
(10) Pub. No.: US 2006/0020328 A1
(43) Pub. Date: Jan. 26, 2006

(54) Composite vascular graft having bioactive agent

(76) Inventor: Sharon Mi Lyn Tan, Allston, MA (US)

Correspondence Address: HOFFMANN & BARON, LLP, 6900 JERICHO TURNPIKE, SYOSSET, NY, 11791, US

(21) Appl. No.: 10/898,102

(22) Filed: Jul. 23, 2004

(51) Int. Cl. A61F 2/06

(52) U.S. Cl. 623/1.42

(57) ABSTRACT

A composite vascular graft incorporates bioactive agents to deliver therapeutic materials and/or inhibit or reduce bacterial growth during and following the introduction of the graft to the implantation site in a vascular system. A composite vascular graft includes a porous tubular graft member. A flexible ePTFE sheath is disposed over the tubular graft member. The sheath includes the bioactive agents incorporated therein.

### FIELD OF THE INVENTION

The present invention relates to implantable medical devices which inhibit or reduce bacterial growth during their use in a living body. More particularly, the present invention relates to composite vascular grafts which incorporate bioactive agents to deliver therapeutic materials and/or to inhibit or reduce bacterial growth during and following the introduction of the graft to the implantation site in the body.

### BACKGROUND OF THE INVENTION

In order to repair or replace diseased or damaged blood vessels it is well known to use implantable vascular grafts in the medical arts. These vascular grafts, which are typically polymeric tubular structures, may be implanted during a surgical procedure or maybe interluminally implanted in a percutaneous procedure.

Such medical procedures employing vascular grafts introduce a foreign object into a patient's vascular system. Therefore, the risk of infection must be addressed in any such procedure.

Vascular graft infection is reported to occur in from about 1% to 6% of the procedures. More significantly, vascular graft infections are associated with a high mortality rate of between 25% to 75%. Moreover, morbidity rates for vascular graft infections are in the range of between 40% and 75%. Infections caused by vascular grafts are also known to prolong hospital stays, thereby greatly increasing the cost of medical care.

Numerous factors contribute to the risk of vascular graft infection. Such factors include the degree of experience of the surgeon and operating room staff. The age of the patent and the degree to which the patient is immunocompromised also are strong risk factors with respect to vascular graft insertion. Other common factors associated with vascular graft infection risks include sterility of the skin of the patient, as well as the materials being implanted.

It has been found that the mechanism of infection for many implanted devices is attributed to local bacterial contamination during surgery. Bacteria on the device produce an extracellular slime matrix/biofilm during colonization, which coats the polymer surface. This biofilm protects the bacteria against the patient's defense mechanisms. The biofilm layer also reduces the penetration of antibiotics.

The most common infectious agents are: staphylococcus aureus, pseudomonas aeruginosa, and staphylococcus epidermis. These agents have been identified in over 75% of all reported vascular infections. Both staphylococcus aureus and pseudomonas aeruginosa, show high virulence and can lead to clinical signs of infection early in the post-operative period (less than four months). It is this virulence that leads to septicemia and is one main factor in the high mortality rates. Staphylococcus epidermis is described as a low virulence type of bacterium. It is late occurring, which means it can present clinical signs of infection up to five years post-operative. This type of bacterium has been shown to be responsible for up to 60% of all vascular graft infections. Infections of this type often require total graft excision, debridement of surrounding tissue, and revascularization through an uninfected route.

Such high virulence organisms are usually introduced at the time of implantation. For example, some of the staphylococcus strains (including staphylococcus aureus) have receptors for tissue ligands such as fibrinogen molecules which are among the first deposits seen after implantation of a graft. This tissue ligand binding provides a way for the bacteria to be shielded from the host immune defenses as well as systemic antibiotics. The bacteria can then produce polymers in the form of a polysaccharide that can lead to the aforementioned slime layer on the outer surface of the graft. In this protective environment, bacterial reproduction occurs and colonies form within the biofilm that can shed cells to surrounding tissues (Calligaro, K. and Veith, Frank, Surgery, 1991 VI 10-No. 5, 805-811). Infection can also originate from transected lymphatics, from inter-arterial thrombus, or be present within the arterial wall.

There are severe complications as a result of vascular graft infections. For example, anastonomic disruption due to proteolytic enzymes that the more virulent organisms produce can lead to a degeneration of the arterial wall adjacent to the anastomosis. This can lead to a pseudoaneurism which can rupture and cause hemodynamic instability. A further complication of a vascular graft infection can be distal styptic embolisms, which can lead to the loss of a limb, or aortoenteric fistulas, which are the result of a leakage from a graft that is infected and that leads to gastrointestinal bleeding (Greisler, H., Infected Vascular Grafts. Maywood, Ill., 33-36).

Desirably, it would be beneficial to prevent any bacteria from adhering to the graft, or to the immediate area surrounding the graft at the time of implantation. It would further be desirable to prevent the initial bacterial biofilm formation described above by encouraging normal tissue ingrowth within the tunnel, and by protecting the implant itself from the biofilm formation.

Silver is an antiseptic agent that has been shown in vitro to inhibit bacterial growth in several ways. For example, it is known that silver can interrupt bacterial growth by interfering with bacterial replication through a binding of the microbial DNA, and also through the process of causing a denaturing and inactivation of crucial microbial metabolic enzymes by binding to the sulfhydryl groups (Tweten, K., J. of Heart Valve Disease 1997, V6, No. 5, 554-561). It is also known that silver causes a disruption of the cell membranes of blood platelets. This increased blood platelet disruption leads to increased surface coverage of the implants with platelet cytoskeletal remains. This process has been shown to lead to an encouragement of the formation of a more structured (mature state) pannus around the implant. This would likely discourage the adhesion and formation of the biofilm produced by infectious bacteria due to a faster tissue ingrowth time (Goodman, S. et al, 24^{th} Annual Meeting of the society for Biomaterials, April 1998, San Diego, Calif.; pg. 207).

It is known to incorporate antimicrobial agents into a medical device. For example, prior art discloses an ePTFE vascular graft, a substantial proportion of the interstices of which contain a coating composition that includes: a biomedical polyurethane; poly(lactic acid), which is a biodegradable polymer; and the anti-microbial agents, chlorhexidine acetate and pipracil. The prior art further describes an ePTFE hernia patch which is impregnated with a composition including silver sulfadiazine and chlorhexidine acetate and poly(lactic acid).

It is also known to provide a device, such as a stent or vascular prosthesis, including an overlying biodegradable coating layer that contains a drug. The coating layer includes an anti-coagulant drug, and, optionally, other additives such as an antibiotic substance.

Further prior art describes a medical implant wherein an antimicrobial agent penetrates the exposed surfaces of the implant and is impregnated throughout the material of the implant. The medical implant may be a vascular graft and the material of the implant may be polytetrafluoroethylene (PTFE). The antimicrobial agent is selected from antibiotics, antiseptics and disinfectants.

Furthermore, prior art is known, which discloses that silver can be deposited onto the surface of a porous polymeric substrate via silver ion assisted beam deposition prior to filling the pores of the porous polymeric material with an insoluble, biocompatible, biodegradable material. The patent further discloses that antimicrobials can be integrated into the pores of the polymeric substrate. The substrate may be a porous vascular graft of ePTFE.

It is also known to provide an anti-infective medical article including a hydrophilic polymer having silver chloride bulk distributed therein. The hydrophilic polymer may be a laminate over a base polymer. Preferred hydrophilic polymers are disclosed as melt processible polyurethanes. The medical article may be a vascular graft. A disadvantage of this graft is that it is not formed of ePTFE, which is known to exhibit superior biocompatibility and to have natural antithrombogenic properties. The ePTFE material has a microporous structure defined by nodes interconnected by fibrils, which facilitates a degree of tissue ingrowth while remaining substantially fluid-tight.

Moreover, prior art describes an implantable medical device that can include a stent structure, a layer of bioactive material posited on one surface of the stent structure, and a porous polymeric layer for controlled release of a bioactive material which is posited over the bioactive material layer. The thickness of the porous polymeric layer is described as providing this controlled release. The medical device can further include another polymeric coating layer between the stent structure and the bioactive material layer. This polymeric coating layer is disclosed as preferably being formed of the same polymer as the porous polymeric layer. Silver can be included as the stent base metal or as a coating on the stent base metal. Alternatively, silver can be in the bioactive layer or can be posited on or impregnated in the surface matrix of the porous polymeric layer. Polymers of polytetrafluoroethylene and bioabsorbable polymers can be used. A disadvantage of this device is that the porous polymeric outer layer needs to be applied without the use of solvents, catalysts, heat or other chemicals or techniques, which would otherwise degrade or damage the bioactive agent deposited on the surface of the stent.

Further prior art describes an antimicrobial vascular graft made with a porous antimicrobial fabric formed by fibers which are laid transverse to each other, and which define pores between the fibers. The fibers may be of ePTFE. Ceramic particles are bound to the fabric material, the particles including antimicrobial metal cations thereon, which may be silver ions. The ceramic particles are exteriorly exposed and may be bound to the graft by a polymeric coating material, which may be a biodegradable polymer. A disadvantage of this device is that the biodegradable coating layer does not provide sufficient tensile strength for an outer graft layer. Moreover, this graft does not include a polymeric ePTFE tube, which has desirable properties for a vascular graft, as described above.

There is a need for additional antimicrobial vascular grafts formed of ePTFE. In particular, there is a need for ePTFE multi-layered vascular grafts which incorporate antimicrobial agents and/or multiple thrombogenic agents that can be controllably released from non-biodegradable materials in the graft to suppress infection following implantation and to prevent biofilm formation. It would also be desirable to provide such grafts with sufficient tensile strength in the tissue-contacting outer layer and with good cellular communication between the blood and the perigraft tissue in the luminal layer.

### SUMMARY OF THE INVENTION

The present invention provides a composite vascular graft which incorporates bioactive agents which can be delivered to the implantation site. The composite vascular graft of the present invention includes a porous tubular graft member. The porous tubular graft member is covered with a flexible ePTFE sheath which has incorporated therein the bioactive agents. The ePTFE sheath exhibits sufficient elasticity to permit placement over the tubular graft member. The sheath may be an extruded tube or from an extruded sheet which is wrapped around the tubular graft member.

The present invention also provides a method for forming a composite vascular graft which incorporates bioactive agents therein. A porous tubular graft member is provided. The porous tubular graft member is covered with a flexible ePTFE sheath having bioactive agents incorporated therein. The porous tubular graft member may be covered by extruding the ePTFE sheath in a tubular configuration and placing the ePTFE sheath over the graft member. Alternatively, the tubular graft member may be covered by extruding the ePTFE sheath in a sheet like configuration and wrapping the sheet about the tubular graft member. The bioactive agent may be extruded with the extrusion of the sheath.

### BRIEF DESCRIPTION OF THE DRAWINGS

FIG. 1 is a perspective showing a partial insection of a composite vascular graft of the present invention.

FIG. 2 is a cross-sectional showing of an embodiment of a stent/graft composite of the present invention.

FIG. 3 is a perspective showing a partial insection of an extruded ePTFE tubular graft member used in combination with the composite vascular graft of FIG. 1.

FIG. 4 is a perspective showing a partial insection of a textile porous tubular graft member used in combination with the composite vascular graft of FIG. 1.

FIG. 5 is a perspective showing of an extruded ePTFE tube used in combination with the composite vascular graft in FIG. 1.

FIG. 6 is a perspective showing of an extruded sheath used in combination with the composite vascular graft of FIG. 1.

### DETAILED DESCRIPTION OF THE INVENTION

In preferred embodiments of the present invention, the implantable composite device is a multi-layered tubular structure which is particularly suited for use as an arterial-venous (AV) graft. The prosthesis preferably includes at least one tubular graft member made of a textile and/or ePTFE. Furthermore, the prosthesis preferably includes a very thin ePTFE tube sheath wrapping the vascular graft which is non-biodegradable and designed to regulate delivery of an antimicrobial agent associated therewith to the site of implantation.

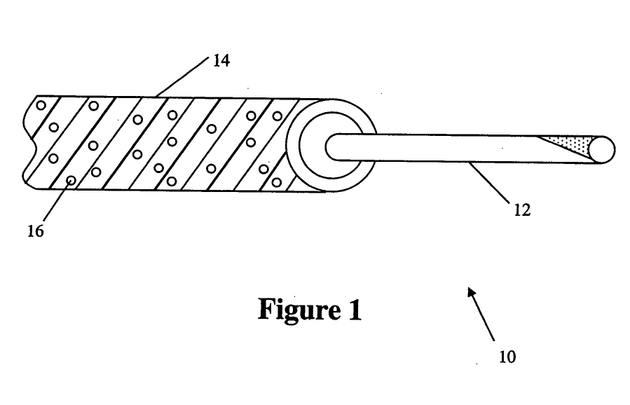

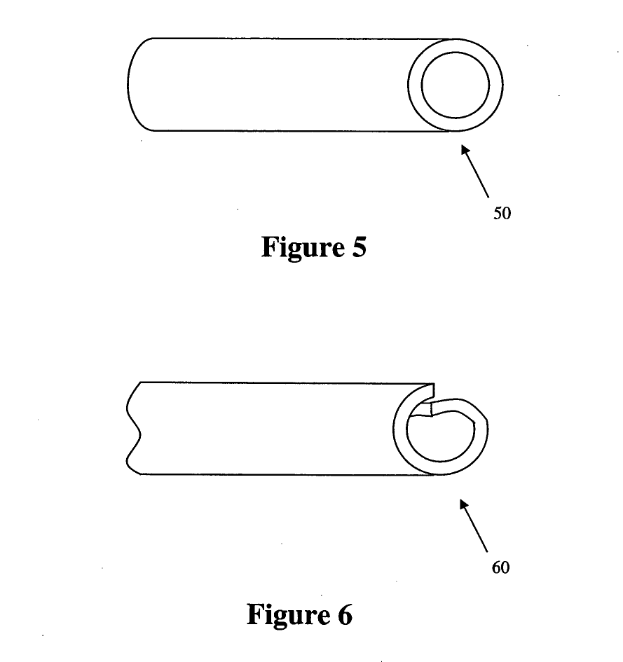

FIG. 1 shows vascular graft 10 of the present invention. As noted above, the present invention takes the preferred embodiment of a tubular graft of composite structure. However, it may be appreciated that the present invention also contemplates other implantable multi-layer prosthetic structures such as vascular patches, blood filters, film wraps for implantable devices such as stents, hernia repair fabrics and plugs and other such devices where such structures may be employed. As shown in FIG. 1, the composite device 10 of the present invention includes a tubular vascular graft member 12 which is made of a textile and/or ePTFE. An ePTFE sheath 14 covers the graft member 12. The ePTFE sheath 14 may preferably be extruded in a tubular configuration and is placed over the graft member 12, as will be described in further detail with reference to FIG. 5. Alternatively, the ePTFE sheath 14 is extruded in a sheet-like configuration and wrapped about the tubular graft member 12, as will be described in further detail with reference to FIG. 6. The ePTFE sheath 14 is flexible and slightly elastic in nature to allow the wrap to be placed on top of the vascular graft 12. For a textile vascular graft, this eliminates the need for a collagen coating since textile grafts are permeable. The ePTFE sheath 14 is a very thin layer of ePTFE which can be extruded and expanded with antimicrobial agents and/or multiple thrombogenic agents 16 other like drugs to address different disease states of an implanted vascular graft. These bioactive agents 16 are preferably distributed substantially evenly throughout the bulk of the ePTFE sheath 14 as will be described in greater detail below.

The bioactive agents may include antimicrobial or antibiotic agents. The antibiotic agents are of the type selected from the group consisting of ciprofloxacin, vancomycin, minocycline, rifampin and other like agents.

The antimicrobial agents include antiseptic agents selected from the group consisting of silver, chlorhexidine, triclosan, iodine, benzalkonium chloride and other like agents.

These antimicrobial or antibiotic agents 16 can be used alone or in combination of two or more of them. These agents 16 are dispersed throughout the ePTFE sheath 14. The amount of each antimicrobial or antibiotic agent 16 used to impregnate the ePTFE sheath 14 varies to some extent, but is at least of an effective concentration to inhibit the growth of bacterial and fungal organisms.

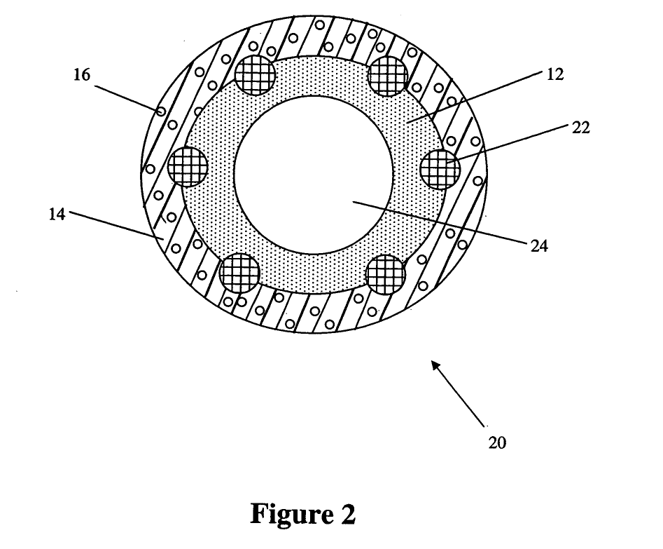

It is well within the contemplation of the present invention that a stent can be interposed between the tubular members of the graft of the present invention. With reference to FIG. 2, a stent/graft composite device 20 of the present invention is shown. Device 20 includes inner vascular graft tubular member 12 and ePTFE sheath 14 covering the graft 12. As described above, the ePTFE sheath 14 incorporates a bioactive agent 16. The ePTFE sheath 14 is flexible and slightly elastic in nature to allow the sheath 14 to be placed on top of the vascular graft 12. Central lumen 24 extends throughout tubular composite graft 20. An expandable stent 22 may be interposed between inner graft tubular member 12 and ePTFE sheath 14. Stent 22, which may be associated with the graft of the present invention, is used for increased support of the blood vessel and increased blood flow through the area of implantation. It is noted that radial tensile strength at the outer ePTFE sheath 14 enables the graft to support, for example, radial expansion of stent 22, when present. In order to facilitate hemodialysis treatment, a significant number of patients suffering from hypertension or poor glycemic control in diabetes will have a synthetic vascular graft surgically implanted between the venous and arterial systems. Typically, these grafts become occluded over time. In these instances, a covered stent across the venous anastomotic site in patients with significant stenosis may aid in prolonging the patency of these grafts, which would avoid painful and typically expensive surgical revisions. For these reasons, it is well within the contemplation of the present invention that a stent covered with or incorporated within the vascular graft of the present invention may be useful for AV access.

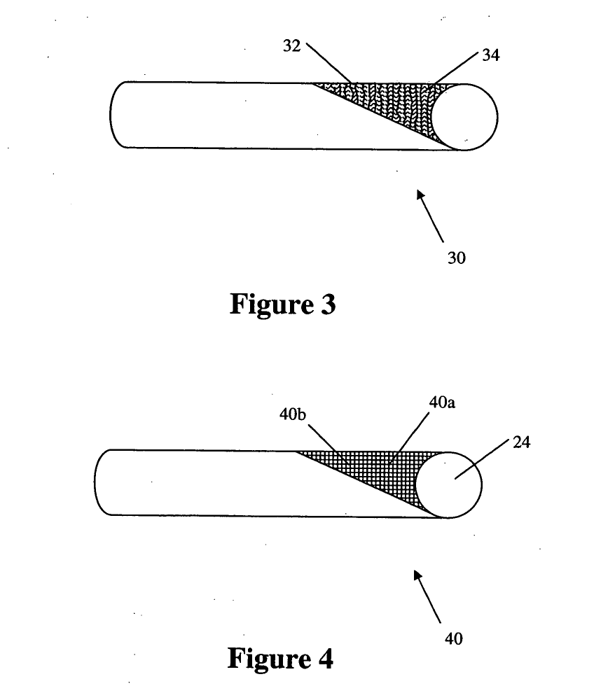

As noted above, in one aspect of the present invention, composite device 10 includes an ePTFE graft member 12 depicted in FIG. 1. PTFE exhibits superior biocompatibility and low thrombogenicity, which makes it particularly useful as vascular graft material. Desirably, the ePTFE layer is a tubular structure 30, as depicted in FIG. 3. The ePTFE material has a fibrous state which is defined by interspaced nodes 32 interconnected by elongated fibrils 34. The space between the node surfaces that is spanned by the fibrils is defined as the internodal distance 36. In the present invention, the internodal distance in the luminal ePTFE layer is desirably about 70 to about 90 microns. When the term “expanded” is used to describe PTFE, i.e. ePTFE, it is intended to describe PTFE which has been stretched, in accordance with techniques which increase the internodal distance and concomitantly porosity. The stretching may be in uni-axially, bi-axially, or multi-axially. The nodes are stretched apart by the stretched fibrils in the direction of the expansion. Methods of making conventional longitudinally expanded ePTFE are well known in the art.

It is further contemplated that the ePTFE may be a physically modified ePTFE tubular structure having enhanced axial elongation and radial expansion properties of up to 600% by linear dimension. The physically modified ePTFE tubular structure is able to be elongated or expanded and then returned to its original state without an elastic force existing therewithin. Additional details of physically-modified EPTFE and methods for making the same can be found in commonly assigned Application Title “ePTFE Graft With Axial Elongation Properties”, assigned U.S. application Ser. No. 09/898,418, filed on Jul. 3, 2001, published on Jan. 9, 2003 as U.S. Application Publication No. 2003-0009210A1, the contents of which are incorporated by reference herein in its entirety.

As noted above, in another aspect of the present invention, composite device 10 includes a textile graft member 40 depicted in FIG. 1. As will be described in further detail below, virtually any textile construction can be used for the graft 12, including weaves, knits, braids, filament windings, spun fibers and the like. Any weave pattern in the art, including, simple weaves, basket weaves, twill weaves, velour weaves and the like may be used. The weave pattern of the textile graft 40 shown in FIG. 4 includes warp yarns 40a running along the longitudinal length (L) of the graft and fill yarns 40b running around the circumference (C) of the graft, the fill yarns being at approximately 90 degrees to one another with fabrics flowing from the machine in the warp direction. A central lumen 24 extends throughout the tubular composite graft 40, which permits the passage of blood through graft 40 once the graft is properly implanted in the vascular system.

Any type of textile products can be used as yarns for a fabric layer. Of particular usefulness in forming a fabric layer for the composite device of the present invention are synthetic materials such as synthetic polymers. Synthetic yarns suitable for use in the fabric layer include, but are not limited to, polyesters, including PET polyesters, polypropylenes, polyethylenes, polyurethanes and polytetrafluoroethylenes. The yarns may be of the mono-filament, multi-filament, spun-type or combinations thereof. The yarns may also be flat, twisted or textured, and may have high, low or moderate shrinkage properties or combinations thereof. Additionally, the yarn type and yarn denier can be selected to meet specific properties desired for the prosthesis, such as porosity and flexibility. The yarn denier represents the linear density of the yarn (number of grams mass divided by 9,000 meters of length). Thus, a yarn with a small denier would correspond to a very fine yarn, whereas a yarn with a large denier, e.g., 1,000, would correspond to a heavy yarn. The yarns used for the fabric layer of the device of the present invention may have a denier from about 20 to about 200, preferably from about 30 to about 100. Desirably, the yarns are polyester, such as polyethylene terephthalate (PET). Polyester is capable of shrinking during a heat-set process, which allows it to be heat-set on a mandrel to form a generally circular shape.

After forming the fabric layer of the present invention, it is optionally cleaned or scoured in a basic solution of warm water. The textile is then rinsed to remove any remaining detergent, and is then compacted or shrunk to reduce and control in part the porosity of the fabric layer. Porosity of a textile material is measured on the Wesolowski scale and by the procedure of Wesolowski. In this test, a fabric test piece is clamped flatwise and subjected to a pressure head of about 120 mm of mercury. Readings are obtained which express the number of mm of water permeating per minute through each square centimeter of fabric. A zero reading represents absolute water impermeability and a value of about 20,000 represents approximate free flow of fluid.

The porosity of the fabric layer is often about 5,000 to about 17,000 on the Wesolowski scale. The fabric layer may be compacted or shrunk in the wale direction to obtain the desired porosity. A solution of organic component, such as hexafluoroisopropanol or trichloroacetic acid, and a halogenated aliphatic hydrocarbon, such as methylene chloride, can be used to compact the textile graft by immersing it into the solution for up to 30 minutes at temperatures from about 15° C. to about 160° C.

Yarns of the fabric layer may be one ply or multi-ply yarns. Multi-ply yarns may be desirable to impart certain properties onto the drawn yarn, such as higher tensile strengths for the porous graft member.

Referring to FIG. 5 of the present invention, there is shown an extruded ePTFE tube 50 used in combination with the composite vascular graft in FIG. 1. Specifically, the extruded ePTFE tube 50 is placed over the graft member 12, thereby covering the graft 12. The process for forming an ePTFE tube may be described as follows.

An ePTFE tube formed preferably by tubular paste extrusion is placed over a stainless steel mandrel. After being placed on the mandrel, the ePTFE is pleated in a plurality of locations. The pleats are formed by folding the ePTFE layer over itself, creating a gathered section of ePTFE material. The gathered sections lengthen the amount of ePTFE material used to form the tube. After pleating, the ends of the ePTFE tube are secured. The ePTFE tube is coated using an adhesive solution of from 1%-15% Corethane®, 2.5 in DMAc. The coated ePTFE tubular structure is then placed in an oven heated in a range from 18° C. to 150° C. for 5 minutes to overnight to dry off the solution. The coating and drying process can be repeated multiple times to add more adhesive to the ePTFE tubular structure. The pleats are folded perpendicular to the axial length of the tube, such that longitudinal expansion of the sheath will cause the pleats to unfold.

Once dried, the ePTFE tubular structure may be longitudinally compressed in the axial direction to between 1% to 85% of its length to relax the fibrils of the ePTFE. The amount of desired compression may depend upon the amount of longitudinal expansion that was imparted to the base PTFE green tube to create the ePTFE tube. Longitudinal expansion and compression may be balanced to achieve the desired properties. This is done to enhance the longitudinal stretch properties of the resultant sheath. The longitudinal compression process can be performed either by manual compression or by thermal compression. Furthermore, the number and length of the pleated regions of the ePTFE layer, are additional factors that can be modified to alter the properties of the resultant sheath.

Alternatively, an ePTFE sheath can be extruded in a sheet-like configuration as shown in FIG. 6. An extruded ePTFE sheath 60 shown in FIG. 6 is used in combination with the composite vascular graft 12 in FIG. 1. Specifically, the ePTFE sheath 60 is wrapped about the graft member 12 to form a cover or liner, thereby covering the graft 12. The ePTFE sheet 60 can be formed by any process well-known in the PTFE forming art. Once the ePTFE sheet 60 is formed, it is wrapped externally about the graft 12 and seamed along the longitudinal axis to form a cover or liner.

Both the preformed ePTFE tube 50 and the preformed ePTFE sheath 60 allow for further expansion once the graft is implanted and radially deployed.

In one of the embodiments of the present invention, it is contemplated that a dry, finely subdivided antimicrobial agent may be blended with the wet or fluid ePTFE material used to form the sheath before the ePTFE solidifies. Alternatively, it is contemplated that air pressure or other suitable means may be employed to disperse the antimicrobial agent substantially evenly within the pores of the solidified ePTFE.

In situations where the antimicrobial agent is insoluble in the wet or fluid ePTFE material, the antimicrobial agent may be finely subdivided as by grinding with a mortar and pestle. Preferably, the antimicrobial agent is micronized, e.g., a product wherein some or all particles are the size of about 5 microns or less. The finely subdivided antimicrobial agent can then be distributed desirably substantially evenly throughout the bulk of the wet or fluid ePTFE layer before cross-linking or cure solidifies the layer.

Furthermore, it is contemplated that a bioactive agent or drug can be incorporated into the ePTFE sheath in the following manner: mixing into an extrudate used to make the ePTFE sheath, a crystalline, particulate material like salt or sugar that is not soluble in a solvent used to form the extrudate; casting the extrudate solution with particulate material; and then applying a second solvent, such as water, to dissolve and remove the particulate material, thereby leaving a porous ePTFE. The ePTFE may then be placed into a solution containing a bioactive agent in order to fill the pores. Preferably, a vacuum would be pulled on the ePTFE to insure that the bioactive agent applied to it is received into the pores.

Alternatively, the ePTFE sheath of the present invention may achieve localized delivery of a bioactive agent to a site where it is needed in a number of ways. For example, the drug may be coated on the outside surface of the ePTFE. The drug may be applied to the outside surface of the ePTFE such as by dipping, spraying, or painting.

It is also contemplated that the bioactive agent or drug may be encapsulated in microparticles, such as microspheres, microfibers or microfibrils, which can then be incorporated into or on the ePTFE sheath. Various methods are known for encapsulating drugs within microparticles or microfibers (see Patrick B. Deasy, Microencapsulation and Related Drug Processes, Marel Dekker, Inc., New York, 1984). For example, a suitable microsphere for incorporation would have a diameter of about 10 microns or less. The microsphere could be contained within the mesh of fine fibrils connecting the matrix of nodes in the ePTFE sheath. The microparticles containing the drug may be incorporated within a zone by adhesively positioning them onto the ePTFE material or by mixing the microparticles with a fluid or gel and flowing them into the ePTFE sheath. The fluid or gel mixed with the microparticles could, for example, be a carrier agent designed to improve the cellular uptake of the bioactive agent incorporated into the ePTFE sheath. Moreover, it is well within the contemplation of the present invention that carrier agents, which can include hyaluronic acid, may be incorporated within each of the embodiments of the present invention so as to enhance cellular uptake of the bioactive agent or agents associated with the device.

The microparticles embedded in the ePTFE sheath may have a polymeric wall surrounding the drug or a matrix containing the drug and optional carrier agents. Due to the potential for varying thicknesses of the polymeric wall and for varying porosities and permeabilities suitable for containing a drug, there is provided the potential for an additional mechanism for controlling the release of a therapeutic agent in a highly regulated manner.

Moreover, microfibers or microfibrils, which may be drug loaded by extrusion, can be adhesively layered or woven into the ePTFE sheath material of a zone for drug delivery.

The bioactive agents which achieve regulated and specific delivery through their association with the composite device of the present invention, may be selected from growth factors, anti-coagulant substances, stenosis inhibitors, thrombo-resistant agents, antibiotic agents, anti-tumor agents, anti-proliferative agents, growth hormones, antiviral agents, anti-angiogenic agents, angiogenic agents, anti-mitotic agents, anti-inflammatory agents, cell cycle regulating agents, genetic agents, cholesterol-lowering agents, vasodilating agents, agents that interfere with endogenous vasoactive mechanisms, hormones, their homologs, derivatives, fragments, pharmaceutical salts and combinations thereof.

In other embodiments, the bioactive agent associated with the composite device of the present invention may be a genetic agent. Examples of genetic agents include DNA, anti-sense DNA, and anti-sense RNA. DNA encoding one of the following may be particularly useful in association with an implantable device according to the present invention: (a) tRNA or RRNA to replace defective or deficient endogenous molecules; (b) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor; (c) cell cycle inhibitors; (d) thymidine kinase and other agents useful for interfering with cell proliferation; and (e) the family of bone morphogenic proteins. Moreover DNA encoding for molecules capable of inducing an upstream or downstream effect of a bone morphogenic protein may be useful.

The bioactive agents which achieve regulated and specific delivery through their association with the composite device of the present invention, may be selected from silver antimicrobial agents, metallic antimicrobial materials, growth factors, anti-coagulant substances, stenosis inhibitors, thrombo-resistant agents, antibiotic agents, anti-tumor agents, anti-proliferative agents, growth hormones, antiviral agents, anti-angiogenic agents, angiogenic agents, anti-mitotic agents, anti-inflammatory agents, cell cycle regulating agents, genetic agents, cholesterol-lowering agents, vasodilating agents, agents that interfere with endogenous vasoactive mechanisms, hormones, their homologs, derivatives, fragments, pharmaceutical salts and combinations thereof.

While the invention has been described in relation to the preferred embodiments with several examples, it will be understood by those skilled in the art that various changes may be made without deviating from the spirit and scope of the invention as defined in the appended claims.

What is claimed is:

1. A composite vascular graft comprising:
a porous tubular graft member; and
a flexible ePTFE sheath disposed over said tubular graft member; said sheath having incorporated therein bioactive agents.

2. The vascular graft of claim 1, wherein said ePTFE sheath is elastic.

3. The vascular graft of claim 1, wherein said sheath is an extruded tube.

4. The vascular graft of claim 1, wherein said sheath is an extruded sheath.

5. The vascular graft of claim 1, wherein said ePTFE sheath is extruded with said bioactive agent.

6. The vascular graft of claim 1, wherein said graft member comprises ePTFE material.

7. The vascular graft of claim 1, wherein said graft member comprises a textile material.

8. The vascular graft of claim 1, wherein said bioactive agents are antimicrobial agents.

9. The vascular graft of claim 8, wherein said antimicrobial agents are antibiotic agents.

10. The vascular graft of claim 9, wherein said antibiotic agents are selected from the group consisting of: ciprofloxacin, vancomycin, minocycline, rifampin and combinations thereof.

11. The vascular graft of claim 8, wherein said antimicrobial agents are antiseptic agents.

12. The vascular graft of claim 11, wherein said antiseptic agents are selected from the group consisting of: silver, chlorhexidine, triclosan, iodine, benzalkonium chloride and combinations thereof.

13. A method of forming a composite vascular graft, said method comprising the steps of:
providing a porous tubular graft member; and
covering said porous graft member with a flexible ePTFE sheath having bioactive agents incorporated therein.

14. The method of claim 13, wherein said covering step includes:
extruding said sheath in a tubular configuration; and
placing said sheath over said graft member.

15. The method of claim 14 wherein said extruded step includes:
extruding said bioactive agent with said tubular configuration.

16. The method of claim 13 wherein said covering step includes:
extruding said sheath in a sheet configuration; and
wrapping said sheath about said tubular graft member.

17. The method of claim 16 wherein said extruded step includes:
extruding said bioactive agent with said sheath configuration.

18. The method of claim 13 wherein said graft member comprises ePTFE material.

19. The method of claim 13 wherein said graft member comprises a textile material.

20. The method of claim 13 wherein said bioactive agents are antimicrobial agents.

21. The method of claim 20 wherein said antimicrobial agents are antibiotic agents.

22. The method of claim 21 wherein said antibiotic agents are selected from the group consisting of: ciprofloxacin, vancomycin, minocycline, rifampin and combinations thereof.

23. The method of claim 20 wherein said antimicrobial agents are antiseptic agents.

24. The method of claim 23, wherein said antiseptic agents are selected from the group consisting of: silver, chlorhexidine, triclosan, iodine, benzalkonium chloride and combinations thereof.
